(19) United States
(12) Patent Application Publication
(10) Pub. No.: US 2026/0096866 A1
(43) Pub. Date: Apr. 9, 2026

(54) DEVICE FOR DOCUMENTING THE USE OF INSERTED IMPLANTS, AND METHOD TO THIS END

(71) Applicant: I.T.S. GmbH, Lassnitzhöhe (AT)

(72) Inventor: Wolfgang AUF, Hart bei Graz (AT)

(73) Assignee: I.T.S. GmbH, Lassnitzhöhe (AT)

(21) Appl. No.: 19/141,981

(22) PCT Filed: Mar. 12, 2024
(86) PCT No.: PCT/AT2024/060086

§ 371 Date: Jun. 20, 2025

(30) Foreign Application Priority Data
Mar. 17, 2023 (AT) A 50203/2023

(51) Int. Cl. A61B 90/00; G06V 10/14

(52) U.S. Cl. CPC A61B 90/361; G06V 10/14; A61B 2090/0805; G06V 2201/03

(57) ABSTRACT

A device and method for documenting a use of implants inserted in an operation. The, implants include an identifier on an implant surface and implants are provided for an operation, The device includes housing with a housing interior in which at least one light source and at least one camera for recording images of a plurality of implants of a surgical set before and after an operation are positioned, and an image evaluating apparatus for evaluating the images recorded before and after the operation by the at least one camera for the purpose of determining the implant or implants inserted during the operation. According to the invention, it is provided that the at least one light source and the at least one camera are configured such that reflections on the implant surfaces are reduced for the purpose of sufficient recognition of the identifiers.

The invention relates to a device for documenting a use of one or more implants inserted in an operation, which implants comprise an identifier on an implant surface and wherein the implant or implants are provided for an operation and can be used in said operation, comprising a housing with a housing interior in which at least one light source and at least one camera for recording images of a plurality of implants of a surgical set before and after an operation are positioned, as well as an image evaluating apparatus for evaluating the images recorded before and after the operation by the at least one camera for the purpose of determining the implant or implants inserted during the operation.

The invention furthermore relates to a method for documenting a use of one or more implants inserted in an operation, which implants comprise an identifier on an implant surface and wherein the implant or implants are provided for an operation and can be used in said operation.

Medical engineering has developed a plurality of implants for various operations, which implants make it possible to treat various fractures of bones or even completely or partially replace joints affected by wear, such as knee or hip joints in particular. The individual implants can have differing complexity and, for example, range from simple screws to elaborate, multi-part implants with the ability to displace and fix individual components.

In medical operations, there are already efforts to optically track surgical instruments during an operation, as has become known from US 2013/0113929 A1. In said document, a system with multiple stationary cameras is disclosed, which cameras cover different regions of an operating room and are connected to a data processing apparatus via which, with the aid of the camera images, individual positions of the parts of the surgical instrument set can be localized, which is then used to be able to instruct the surgical staff accordingly where necessary. An operation is intended to thus become more efficient.

In US 2015/0250551 A1, a method and a device for detecting a loss of individual parts of a surgical instrument set is disclosed. For this purpose, tray with a plurality of regions is provided for the surgical instruments that are to be stored. Identifiers are provided in the tray, for example in the form of a bar code, for each part of a surgical instrument set that is to be stored. The tray can be scanned using a corresponding apparatus having at least one camera, so that it is possible to check whether one or more parts of the surgical instrument set are missing with the aid of an image evaluation.

For implants, there are, for different reasons, also attempts to be able to identify said implants during an operation; among other things, namely for regulatory reasons, as is addressed in DE 699 21 864 T2.

It is desirable that, even long after an operation, it be possible to determine exactly which implant or implants were inserted into a human body. This can be necessary, for example, in order to be able to perform a replacement in a timely manner if need be in the event of specific faulty lots. Another aspect is that implants inserted into or onto the human or animal body can be exposed to corrosion even when fixed only temporarily in the body, whereby substances are released in the human body and can possibly remain in the body, even if the implant has already been removed again. Despite this, there are still just few studies on the effects on the human or animal body of a corrosive degradation of implants having such elements. With a thorough documentation, it would be possible to create a suitable basis for long-term studies in particular.

Yet another aspect of a desirable, accurate documentation is legal issues that arise in the event of implant failure, for example. For liability questions, it can thereby be critical to be able to associate an implant with a specific lot of a producer of the implant.

In the Applicant's WO 2021/217189 A1, a method and a device for a suitable documentation of implants have already become known. A surgical set with implants is thereby provided which is scanned using a camera and a light source so that identifiers on the individual implants can be recorded and subsequently identified using a suitable image evaluation. This process is carried out before an operation and after said operation. Which implant or implants were affixed in or on the person undergoing surgery follows from the documentation of the corresponding difference. Essentially, the device and the method function reliably. The ability of the camera to be moved together with a light source basically ensures suitable conditions so that high-quality images can be recorded by the camera, whereby an exact association is enabled in the image processing. However, in the case of round surfaces such as screw heads, recognition problems can arise in the recognition of the identifiers, which normally comprise many numbers and/or letters.

Starting from the prior art, the object of the invention is to further develop a device of the type named at the outset such that, with a simple design, said device allows a reliable recognition of identifiers of a plurality of implants.

A further object of the invention is to specify a method of the type named at the outset which can be carried out using simpler means without sacrificing a reliability of the documentation of the inserted implant or implants.

The object of the invention is attained if, with a device of the type named at the outset, the at least one light source and the at least one camera are configured such that reflections on the implant surfaces are reduced for the purpose of sufficient recognition of the identifiers.

With the device according to the invention, a reliable recognition of identifiers can be achieved.

As a result of the envisioned reduction of reflections, it is possible that identifiers of individual implants can be recognized with very high reliability. The at least one light source and the at least one camera, preferably multiple light sources and multiple cameras, are thereby coordinated with one another such that, compared with a direct illumination of the implants by the at least one light source, reflections are reduced. The at least one camera, preferably multiple cameras, can thereby be arranged in a movable, or possibly also fixed, manner. A combination of movable cameras together with cameras positioned in a fixed manner is also possible. The at least one light source, preferably a plurality of light sources, can be arranged such that the implant surfaces that are to be recorded by the camera or cameras are not directly illuminated. As a result, reflections can be reduced, in particular minimized. The reduction of reflections thereby refers to a situation in which an implant is illuminated from above with an equivalent light source, as is known from the prior art, for example according to the cited document WO 2021/217189 A1, according to which a camera is moved together with a light source. Thus, according to the invention, the at least one light source and the at least one camera are configured such that reflections on the implant surfaces are reduced. This desired reduction takes place at the expense of an illumination of the implant surfaces, which is, in turn, unfavorable for the recognition of the identifiers. According to the invention, through the reduction of the reflections and the accompanying lower illumination of the implant surface, a suitable balance is created in that, for a given recognizability of the identifiers, the reflections are sufficiently reduced. In other words, it is also possible to consider a favorable balance between sufficient illumination with minimized reflections.

An image evaluation occurs using methods known from the prior art. The at least one camera is connected to a data processing system in a manner suitable for this purpose, for example by wire or via a wireless connection. The recorded images are analyzed in the data processing system in order to ascertain the identifier of each implant and, preferably, associate it with a specific position on the surgical set.

The image evaluation can take place directly in or on the device if the device is equipped with corresponding means. It is also possible that the image evaluation occurs via a server arranged remotely. For this purpose, the device can be connected to the server wirelessly or by fixed lines. It is preferred that the device is equipped with a data processing apparatus which processes the rapid recognition of the identifiers on-site, for example a laptop or a tablet computer. The data processing apparatus can be externally integrated into the device on the housing in order to enable simple operation.

The device is typically embodied such that it can be transported by hand. Typical dimensions depend on the surgical sets that are to be inspected and, for example, can be 50 cm×35 cm×35 cm in terms of length, width and height.

Even though a single light source is sufficient, multiple light sources can advantageously be provided. The light sources can be arranged in the housing interior, for example at least partially with a roughly equal spacing from one another.

If multiple light sources are provided, they can preferably be formed from LED light sources. If the light sources are present as LED light sources, they can be arranged as strips. Typically, the LED strips are arranged in a rectangle corresponding to a housing interior. The LED strips can thereby be arranged adjacently to an inner side of the housing interior defined by housing parts. As a result, an illumination can take place from an inner wall of the housing towards the actual housing interior. The LED strips, or possibly other light sources, are thereby preferably arranged in a plane that lies below that plane in which the surgical set is inserted. An indirect illumination of the implant surfaces is thus automatically achieved, since the implant surfaces face away from the emission direction of the light sources.

The at least one light source can be a point-shaped light source. Alternatively, the at least one light source can also be embodied to be elongate. If multiple light sources are provided, they are advantageously embodied to either each be point-shaped or each be line-shaped or each be strip-shaped, so that the most homogeneous possible lighting conditions in the housing interior are obtained. The specific type of light source is not critical; an indirect illumination of the implant surfaces should be ensured.

In principle, one camera can be sufficient. If, however, an entire cross section of the housing interior or an entire surgical set is to be covered, multiple cameras are advantageously provided, the fields of view of which preferably overlap. With multiple cameras that are arranged or aligned with overlapping fields of view, a high quality of the output images that are to be processed or analyzed can be obtained for the subsequent image processing. Since the cameras record implants from different perspectives in the overlap regions, it is possible to take into account the fact that the implants can have a differing height, that the identifiers can be placed at different positions, and/or that individual implant surfaces can also be tilted relative to an optical camera axis. The more the individual fields of view of individual cameras overlap, the better can be a result of the image evaluation, and therefore of the reliability of the method. Expediently, every single region of an image acquisition for the documentation can be covered by at least two cameras, preferably three or more cameras. An upper limit results from the utility for the subsequent image processing. If three or four cameras cover a single region over the associated field of view, it is possible to obtain very good and reliable results.

Preferably, for surgical sets of a common size, two or three cameras are provided which are arranged at the top end in the housing interior. The cameras can be movably arranged. If this is the case, an actuating motor is provided for moving the cameras, which actuating motor is connected to the cameras in order to move them. The actuating motor is thereby configured such that the cameras can be moved together along a longitudinal axis of the device. The cameras are thereby preferably moved such that they respectively record images at predetermined positions along the longitudinal axis. For example, with a set of three cameras, the full area of a housing interior along the length and width can be captured by four recordings along the longitudinal axis, at the noted predetermined positions. The individual recorded images of the cameras along the longitudinal axis thereby overlap. It is thus possible to obtain a complete, clear image of the area in a length and width direction for the image evaluation. The cameras are thereby moved from one predetermined position to the next. In principle, however, it is also possible that the cameras move continuously at a specific speed and thereby generate recorded images, wherein corresponding images are then evaluated.

The at least one camera can be aligned such that the optical axis thereof is perpendicular to the surgical set, which is normally embodied to be flat, at least to a large extent. The cameras are arranged in a suitable quantity and at a suitable distance from one another so that the regions of the surgical set in which implants are arranged are completely covered, preferably with the noted overlap of the fields of view. An inclination of the optical axis of the cameras different from a position perpendicular to the surgical set is also possible.

For a simplest possible setup, it is preferred that the optical axis of the at least one camera or the optical axes of multiple cameras are respectively perpendicular to that plane in which the surgical set is inserted. The line of sight of a camera is thus perpendicular to the surgical set. The light sources are, as noted, preferably arranged below the surgical set and the related insertion area.

The at least one light source is preferably arranged in a stationary manner. This means that, in the housing interior, the light source(s) cannot be moved, nor can the position thereof be altered in any other manner. It can be provided that the light source(s) can be swiveled in order to change or to optimize the illumination settings and the fields of view. However, it is preferred for the light source(s) that it is/they are configured one time and can then be neither moved or swiveled. In principle, the at least one camera, or possibly multiple cameras, cannot be swiveled during operation, but can be embodied to be able to swivel in order to be suitably adjusted for ongoing operation.

In a particularly preferred variant, the at least one light source is positioned for the indirect illumination of the implants. The at least one light source can be arranged below the insertion area for the surgical set, and thus illuminates from below the implant surfaces that are captured by the camera or the cameras. As a result of this indirect illumination, the at least one light source and the at least one camera are configured such that reflections on the implant surfaces are reduced for the purpose of sufficient recognition of the identifiers. Through a corresponding positioning of the at least one light source in the housing interior, reflections on the implant surfaces can be prevented or at least reduced. Such reflections are particularly disruptive and can result in it no longer being possible to determine an identifier. This is significantly reduced by the indirect illumination. It is thereby advantageously provided that the at least one light source is positioned with an emission direction against a field of view of the at least one camera. In addition, components for generating scattered light can be arranged in the housing interior. The more diffuse the generated light that falls on the surface on an implant is, the fewer reflections occur. This can also be achieved by a suitable adaptation of an inner surface of the housing, for example in that said housing is embodied with a matte surface having a high roughness. For this purpose, for example, a lining of a brightest possible, preferably white, plastic is suitable which has a suitable surface roughness, or bright, preferably white, pieces of fabric. In this context, light-absorbing elements can be provided which are arranged in a lateral and/or rear region of the at least one camera. A quality of the recorded images can thus be further improved, which results in an even more reliable recognition of the identifiers by the image evaluation.

Other types of indirect lighting are also possible, for example in that sufficiently diffuse light is generated by a sufficiently strong light source, or possibly multiple light sources, and suitable means, for example by scattering on rough surfaces.

In another variant, it can be provided that two polarizing filters are arranged between the at least one camera and the at least one light source. This also makes it possible to reduce reflections on the implant surfaces in an excellent manner. The polarizing filters can thereby be skewed relative to one another at an angle of 10° to 85°, preferably 50° to 82°, in particular 60°to 80°. The settings can be selected such that, on the one hand, an amount of light falling on the camera is optimized, yet, on the other hand, reflections on the implant surfaces are reduced, in particular minimized. Thus, a balanced harmony between the light striking the camera and the reflection reduction or minimization is reached.

The housing is preferably embodied with a drawer which receives the surgical set. The drawer can thereby be embodied such that the surgical set can be inserted into a recess of the drawer. It can thus be ensured that the surgical set is correctly in position. For this purpose, the recess is essentially embodied such that it corresponds to an outer diameter of the surgical set, wherein the surgical set rests in the drawer with an edge at the top end. The implants are arranged on the surgical set such that they are loose or releasably fastened. An implant within the meaning of the invention is thereby that part which can be inserted onto and/or into the human or animal body during an operation, in particular to treat a fracture or the like. These can also be relatively small individual parts such as bone screws or screws for fastening bone plates. However, they can also be bone plates, intramedullary nails, wires or other, normally metal, parts which are used to treat fractures, for corrective operations, or for other purposes.

The housing is advantageously embodied with outer housing parts with which the housing interior can be completely closed. A complete closure of the housing interior can be important in order to prevent reflections. If light were to enter from the outside, this could result in undesired reflections on the implant surfaces.

The housing can thereby be embodied such that a receiving opening for the surgical set is present, wherein the opening can be closed by a drawer that accommodates the surgical set. The drawer can thereby be mounted on suitable slide rails so that the drawer can be easily opened and shut. Aside from the opening for the drawer, the housing interior can also be completely closed off from light entering from the outside. Thus, there remains only the possibility of an entry of light in the region of the opening. In order to eliminate this to the greatest possible extent for ongoing operation, suitable seals can be provided in the region of the opening and/or of the drawer.

The other object of the invention is attained if, with a method of the type named at the outset, implants are provided which are, at least in some regions, coated on an implant surface and a device according to the invention is used.

With a method according to the invention, the advantage is obtained that, with simple means, a precise documentation of implants inserted in an operation can take place. A device according to the invention is used for this purpose. A device of this type is configured to be suitable for carrying out the method. Since the implant surfaces are also coated, the advantages of a device according to the invention have a particular effect. In a method according to the invention, the reflections on the implant surfaces are thus reduced, in particular minimized, for the purpose of sufficient recognition of the identifiers. To this end, the at least one light source and the at least one camera are suitably positioned. This applies accordingly if multiple light sources and/or multiple cameras are provided.

In a further aspect, the invention relates to a use of surface-coated implants for documenting a use of one or more implants inserted in an operation by means of image recognition. It has been shown that, in the case of surface-coated implants, a recording of images using a camera and a subsequent image recognition by means of suitable image-processing software can be carried out with high quality, so that a precise documentation of a use of implants is possible.

The advantages of the concept according to the invention are applied in particular if surface-coated implants are inspected in a device according to the invention. A method according to the invention in particular is applied thereby. Through a suitable positioning of the at least one light source on the one hand and of the at least one camera on the other hand, an amount of disruptive reflections from incident light can already be reduced. If surface-coated implants are provided at the same time, this additionally contributes to a reduction of undesired reflections of light. Thus, viewed conversely, higher light intensities can be used without undesired reflections occurring which would jeopardize the recognizability of the identifiers.

Additional features, advantages, and effects of the invention follow from the exemplary embodiments described below. In the drawings which are thereby referenced:

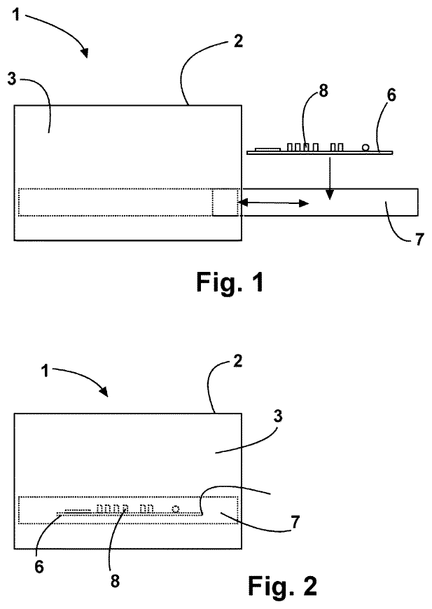

FIG. 1 shows a schematic illustration of a device with a pulled-out drawer;

FIG. 2 shows a schematic illustration of the device from FIG. 1 with a pushed-in drawer;

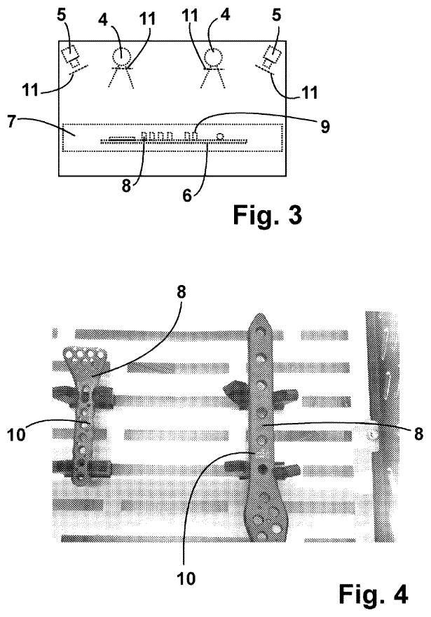

FIG. 3 shows a first variant of a device according to the invention;

FIG. 4 shows implants of a surgical set;

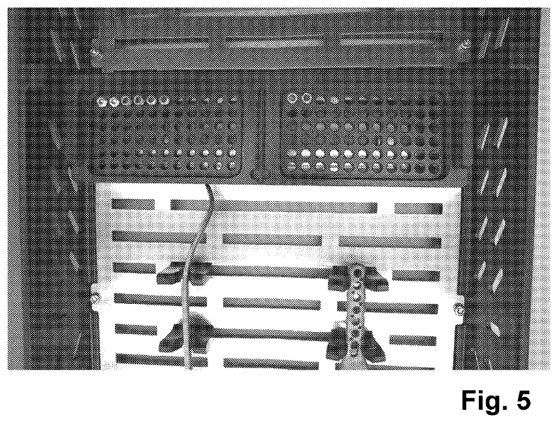

FIG. 5 shows lighting conditions during a recording using the device according to FIG. 3;

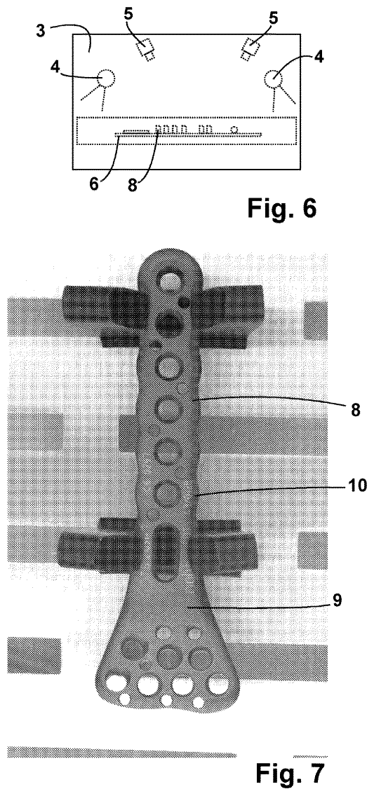

FIG. 6 shows a further variant of a device according to the invention;

FIG. 7 shows an implant and lighting conditions during a recording using a device according to FIG. 6;

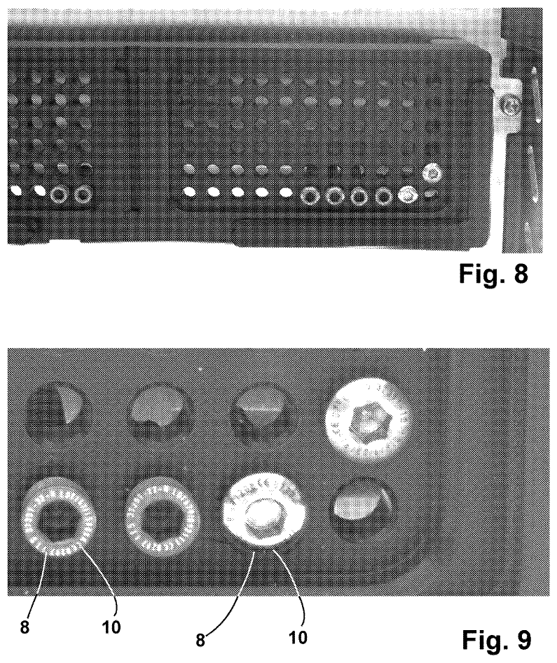

FIG. 8 shows individual implant screws during a recording using a camera;

FIG. 9 shows an enlarged section of the photograph in FIG. 8;

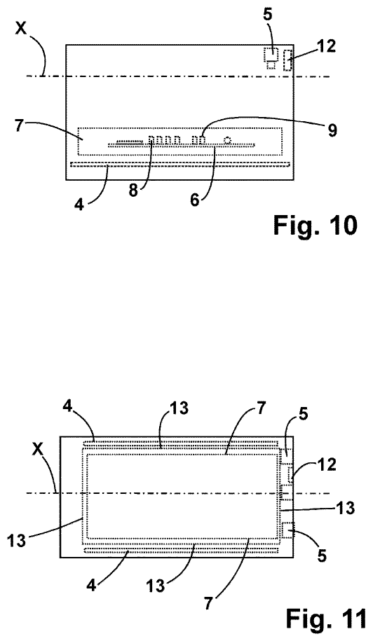

FIG. 10 and FIG. 11 show a schematic illustration of a further variant of a device according to the invention.

In FIG. 1, a device 1 according to the invention can be seen in a highly schematized manner. The device 1 comprises on the outside a housing 2 which, except for an opening for a drawer 7 that will be explained below, is at least essentially, preferably completely, closed so that, basically, no light can enter the housing interior 3. The drawer 7 is mounted, in particular movably mounted, in the housing 2. For this purpose, the housing 2 has a sufficient opening through which the drawer 7 can be pulled out. A surgical set 6 with implants arranged thereon can be inserted into the drawer 7. The drawer 7 can then be pushed into the housing interior 3, wherein a face of the drawer 7 closes the housing 2. For this purpose, the drawer 7 can also be embodied with a sufficiently widened edge that comes to rest on the outside of the housing 2 and simultaneously ensures that the drawer 7 cannot be moved any further. A pushed-in state of the drawer 7 is illustrated in FIG. 2.

The situations illustrated in FIG. 1 and FIG. 2 generally show that the drawer 7 with a surgical set 6 and implants 8 arranged thereon is located entirely inside of the housing 2, and that said housing 2 is essentially closed such that no light can penetrate into the housing interior 3 from the outside. The device 1 does not necessarily need to be equipped with a drawer 7. It is also possible that the surgical set 6 is introduced into the housing interior 3 from above. In this case, a lid is provided which closes the housing 2 at the top end as an outer housing part. Other types of introduction of the surgical set 6 into the housing interior 3 are also conceivable. What is important is that, after the introduction of the surgical set 6 with the implants 8 placed thereupon or arranged thereon, essentially no light can enter the housing interior 3. These statements about a possible design of the housing 2 apply generally, and also specifically to the embodiments below.

In FIG. 3, a first variant of a device 1 according to the invention is illustrated. In addition to the housing 2 which closes the housing interior 3, together with a drawer 7 located therein, which drawer 7 carries the surgical set 6 and implants 8, multiple cameras 5 and light sources 4 are provided. Polarized filters 11 are respectively located in front of both the light sources 4 and the cameras 5. The polarized filters 11 are set such that light that is initially emitted by the light sources 4 and linearly polarized by the polarizing filters 11, which light falls on the implants 8 and is reflected thereby, is eliminated to a considerable extent. Disruptive reflections on the cameras 5 are thus neutralized. In order to nevertheless obtain a sufficiently significant image, the polarizing filters 11 are not in a relatively skewed state that produces a dark image (90° position of the polarizing filters 11), but rather at an angle of less than 90°, for example 50° to 75°. An optimum can be found with relative ease by varying the respective polarizing filters 11. The cameras 5 are connected to an image processing unit, not shown, which can be integrated inside the housing 2 of the device 1. With the image processing unit, images recorded by the cameras 5 can be processed so that identifiers 10 placed on the implant surfaces 9 can be recognized and stored. However, the image processing unit can, in principle, also be arranged outside the housing 2, for example as a separate unit.

In FIG. 4, a corresponding recording is shown, wherein two implants 8 are arranged on a base. As can be seen, identifiers 10 designed to be relatively small can be recognized well on the implants 8. The images recorded by the cameras 5 can be evaluated by means of image processing such that each implant 8 is uniquely identified. This process is carried out before an operation and after an operation. The evaluation of the corresponding images results, by establishing the difference, in the identification of those implants 8 which were inserted during the operation. It is conceivable that an implant 8 goes missing during the operation and is then erroneously associated with the patient. However, this is not critical, since the implant 8 is not located on or in the patient. All implants 8 located on or in the patient are documented and traceable even years later.

It is particularly beneficial if the implants 8 are surface-coated. This is shown clearly in the example in FIG. 5 for different screws which are visible in the upper right of FIG. 5. Whereas surface-coated implants 8 or screws which have a certain roughness, and the identifiers 10 thereof, can be identified without difficulty by means of camera recordings and image recognition, this is partially or entirely no longer possible for a non-surface-coated screw, since said screw excessively reflects light under the given conditions.

In FIG. 6, a further variant of the device 1 according to the invention is illustrated. In this case, the light sources 4 as well as the cameras 5 are once again positioned in a stationary manner in the housing interior 3. In terms of the emissions characteristics, however, the light sources 4 are positioned such that they face away from the fields of view of the cameras 5 and, therefore, no light that is emitted by the light sources 4 can directly strike a camera 5. Furthermore, individual components, not illustrated in greater detail, in the housing interior 3 are provided which ensure that the light emitted by the light sources 4 is very highly scattered. For this purpose, it can also be provided that an inner surface of the housing 2 is suitably embodied, for example by a very rough surface. This ensures that the light more or less directed by the light sources 4 is very highly scattered, whereby reflections on the implant surfaces 9 are prevented at least to a large extent. This is illustrated by way of example in FIG. 7 for an implant 8 on which the identifier 10 can be seen well, so that a unique identification of the implant 8 is possible before the operation and, if said implant 8 is not inserted, after the operation. An evaluation once again takes place using an image processing unit which can be integrated into the housing 2 and functions as described above. In the device 1 according to FIG. 6, it is also expedient if said device 1 is used in combination with surface-coated implants 8. As can be seen in FIG. 8, non-surface-coated implants 8, here in the form of screws, are still very highly prone to reflection in the case of diffuse incident light. This can be avoided with surface-coated implants 8. Whereas, as the enlarged section in FIG. 9 shows, the identifier 10 is barely still visible for a non-surface-coated screw, this is possible without difficulty for the surface-coated implants 8 or screws.

In FIG. 10 and FIG. 11, a particularly preferred variant of a device 1 according to the invention is illustrated. In contrast to the embodiments explained above, the device 1 is embodied with movable cameras 5, normally two or three cameras 5, which are arranged at the top end in the device 1. The cameras 5 are connected to an actuating motor 12 which moves the cameras 5 synchronously along a longitudinal axis X of the device 1. At predetermined positions, the cameras 5 record images of a surgical set 6 and implants 8 stored therein. As can be seen in FIG. 10, individual light sources 4, embodied in the form of an LED strip, are arranged below a plane in which the surgical set 6 is inserted, and emit light upwards. The surgical set 6 can, for example be introduced into the housing interior 3 using a drawer 7, as was explained above for the other embodiments. An illumination of the implant surfaces 9 thus occurs indirectly from below. In FIG. 11, it can additionally be seen that a core of the housing interior 3 is insulated against the light sources 4 by a light-scattering means 13, for example what is referred to as a photo cloth. Thus, not only is an indirect illumination of the implant surfaces 9 achieved, available light is additionally scattered before entry into the core of the housing interior 3, whereby a diffuse illumination of the surfaces of the implants 8 results. Reflections are thus reduced. If the implants 8 are also surface-coated, reflections can be minimized to such an extent that, even in the case of very complex identifiers, an error-free recognition is possible with the aid of the images recorded by the cameras 5.

1. A device for documenting a use of one or more implants inserted in an operation, which implants comprise an identifier on an implant surface and wherein the implant or implants are provided for an operation and can be used in said operation, comprising a housing with a housing interior in which at least one light source and at least one camera for recording images of a plurality of implants of a surgical set before and after an operation are positioned, as well as an image evaluating apparatus for evaluating the images recorded before and after the operation by the at least one camera for the purpose of determining the implant or implants inserted during the operation, wherein the at least one light source and the at least one camera are configured such that reflections on the implant surfaces are reduced for the purpose of sufficient recognition of the identifiers, wherein multiple cameras are movably arranged and multiple light sources are positioned for the indirect illumination of the implants wherein the light sources are arranged below that plane in which the surgical set is inserted, wherein in that multiple cameras are movably arranged and multiple light sources are positioned for the indirect illumination of the implants, and wherein the light sources are arranged below that plane in which the surgical set is inserted.

2. The device according to claim 1, wherein multiple light sources are provided.

3. The device according to claim 1, wherein the at least one light source is a point-shaped light source

4. The device according to claim 1, wherein multiple cameras are provided, the fields of view of which preferably overlap.

5. The device according to claim 1, wherein the at least one light source and/or the at least one camera are arranged in a stationary manner.

6. (canceled)

7. The device according to claim 1, wherein the at least one light source is positioned with an emission direction against a field of view of the at least one camera.

8. The device according to claim 1, wherein components for generating scattered light are arranged in the housing interior.

9. The device according to claim 1, wherein light-absorbing elements are provided which are arranged in a lateral and/or rear region of the at least one camera.

10. The device according to one claim 1, wherein two polarizing filters are arranged between the at least one camera and the at least one light source.

11. The device according to claim 10, wherein the polarizing filters are skewed relative to one another at an angle of 10° to 85°,preferably 50° to 82°, in particular 60° to 80°.

12. The device according to claim 1, wherein the housing is embodied with outer housing parts with which the housing interior can be completely closed.

13. (canceled)

14. A method for documenting a use of one or more implants inserted in an operation, which implants comprise an identifier on an implant surface and wherein the implant or implants are provided for an operation and can be used in said operation, wherein implants are provided which are coated, at least in some regions, on an implant surface and a device according to claim 1 is used.

15. A use of surface-coated implants for documenting a use of one or more implants inserted in an operation by image recognition.

16. The device according to claim 1, wherein the housing is embodied with a drawer into which the surgical set can be inserted.
